(19) United States
(12) Patent Application Publication
(10) Pub. No.: US 2011/0224451 A1
(43) Pub. Date: Sep. 15, 2011

(54) PROCESS FOR PRODUCTION OF BIODIESEL

(76) Inventors: Rajiv Kumar Chaturvedi, Baner (IN); Nawalkishor Mal, Baner (IN); Kyatanahalli Srinivasa Nagabhushana, Baner (IN); Debabrata Rautaray, Baner (IN); Tushar R. Shinde, Baner (IN)

(21) Appl. No.: 12/997,293

(22) PCT Filed: Jun. 11, 2008
(86) PCT No.: PCT/IN09/00331

§ 371 Date: Feb. 28, 2011

(30) Foreign Application Priority Data
Jun. 11, 2008 (IN) 1239/MUM/2008
Jun. 30, 2008 (IN) 1356/MUM/2008

(51) Int. Cl. C07C 67/08; B01J 27/053; B01J 27/055; B01J 21/06; B01J 21/10; C07C 69/00; C07C 67/03

(52) U.S. Cl. 554/167; 502/217; 502/218; 502/243; 502/250; 502/252; 554/1; 554/170

(57) ABSTRACT

The invention relates to a process for producing alkyl esters, the process comprising reacting a feedstock that includes one or more fatty acid glycerol esters or one or more fatty acid or mixture thereof with a C₁ to C₄ alcohol in the presence of a catalyst at a temperature substantially 100° C. or more, the catalyst including a catalyst composition comprising oxides, mixed oxides, silicates or sulphates of two or more of silica, aluminium, iron, calcium, magnesium, sodium and potassium.

The following disclosure generally relates to a process for production of alkyl esters. More particularly, the disclosure relates to a process for production of fatty acid alkyl esters useful in biofuels by esterification and/or transesterification reaction using a solid catalyst.

### BACKGROUND

Biodiesel is a non-petroleum based fuel that consists of fatty acid alkyl esters, made from vegetable oils or animal fats. The lower alkyl (C₁-C₄) esters generated from the oils and fats can be appropriately blended with petroleum diesel that makes the blend suitable for use in diesel engine. As biodiesel is a biodegradable and non toxic alternative to diesel fuel, it is becoming increasingly useful as a “green fuel”.

Commercial production of biodiesel is carried out by a transesterification reaction of vegetable oil or animal fat (hereafter called feedstock). Vegetable oil or animal fats is reacted with alcohol (e.g., methanol, ethanol) to convert the triglyceride in oils and fats to alkyl esters (biodiesel) and glycerine by-product. Since this reaction is slow, the reaction is carried out in the presence of a catalyst.

Most commercial processes for the production of biodiesel currently use a homogeneous alkali catalyst at 60-65° C. While the homogeneity of the reaction mass enhances the conversion rate, the catalyst is part of the reaction product. This makes it necessary to carry out a complicated step of separation and/or removal of the catalyst. The process of separating biodiesel from catalyst and glycerol involves a neutralization process with strong acids (e.g., HCl), and extensive washes with water to remove the resulting sodium salt. Further, in order to remove sodium chloride from glycerol and to obtain glycerol in high purity, distillation of high boiling glycerol has to be carried out which is an energy intensive operation.

The use of alkali catalyst also cause saponification of free fatty acids contained in fats and oils to form soaps as by products, whereby it becomes necessary to carry out a step of washing with large amounts of water. In addition, the yield of alkly esters (biodiesel) decreases due to the emulsification effect of the soaps generated and, in certain instances, the subsequent glycerine purification process also becomes complicated. In order to overcome the problem associated with free fatty acids, a strong homogeneous acid like sulphuric acid is generally used along with the reactant alcohol (e.g., methanol) as a pre-treatment catalyst that converts free fatty acids to alkyl esters. However, if acid is used in the pre-treatment process, neutralization of oil has to be done before transesterification reaction may be carried out. This further creates economical and environmental concerns.

In order to overcome the problems associated with use of a homogeneous catalyst, heterogenous solid catalysts for the transesterification of oils to biodiesel have been developed. For example, various basic metal oxides, such as magnesium methoxide, calcium oxide, calcium alkoxide, and barium hydroxide, have been demonstrated to be active catalysts for transesterification. However, the recyclability of these solid base catalysts is poor. This is because of the moderate solubility of some of these solid metal oxides, hydroxides and alcoxides in methanol/ethanol and strong physical adsorption of the reaction products on their surfaces.

Use of double metal cyanides and metal (e.g., Zn, Mo) embedded on supports (like alumina) as recyclable solid catalysts have also been claimed recently. The major drawback of such a catalyst is its relatively higher cost of preparation and therefore requiring large number of recycles. These recovery and further activation for recycling of catalyst cause technical and economic restrains.

In view of these drawbacks, there is a need to develop a process for biodiesel production that does not require tedious aqueous washes and neutralization steps. An economical and recyclable catalyst that can be easily separated from the biodiesel products for the conversion of oils to biodiesel is also needed. Moreover a catalyst that can economically catalyse both the esterification of free fatty acids and transesterify oils to biodiesel is desirable.

### SUMMARY

The invention relates to a process for producing alkyl esters, the process comprising reacting a feedstock that includes one or more fatty acid glycerol esters or one or more fatty acid or mixture thereof with a C₁ to C₄ alcohol in the presence of a catalyst at a temperature substantially 100° C. or more, the catalyst including a catalyst composition comprising oxides, mixed oxides, silicates or sulphates of two or more of silica, aluminium, iron, calcium, magnesium, sodium and potassium.

The invention also relates to a process for producing alkyl esters, the process comprising reacting a feedstock that includes one or more fatty acid glycerol ester or one or more fatty acid or mixture thereof with a C₁ to C₄ alcohol in the presence of a catalyst, the catalyst including a nano composite catalyst having a particle size in the range of 5 nm to 1000 nm and comprising of oxides or mixed oxides of one or more of silica, alumina, calcium and iron.

The invention also relates to a process for producing alkyl esters, the process comprising reacting a feedstock that includes one or more fatty acid glycerol esters or one or more fatty acid or mixture thereof with a C₁ to C₄ alcohol in the presence of a catalyst, the catalyst being a catalyst composite comprising of a catalyst composition comprising of oxides, mixed oxides, silicates or sulphates of two or more of silica, aluminium, iron, calcium, magnesium, sodium and potassium; and a nano composite catalyst having a particle size in the range of 5 nm to 1000 nm and comprising of oxides or mixed oxides of one or more of silica, alumina, calcium and iron. The invention also relates to Alkyl esters obtained by the processes described above.

The invention also relates to a catalyst composite for the production of alkyl esters from a feedstock including one or more fatty acid glycerol esters or one or more fatty acids or mixture thereof, wherein the catalyst composite comprises of a nano composite catalyst having a particle size in the range of 5 nm to 1000 nm and comprising of oxides or mixed of one or more of silica, alumina, calcium and iron.

The invention also relates to a catalyst composite for the production of alkyl esters from a feedstock including one or more fatty acid glycerol esters or one or more fatty acids or mixture thereof, wherein the catalyst composite comprises a catalyst composition comprising of oxides, mixed oxides, silicates or sulphates of two or more of silica, aluminium, iron, calcium, magnesium, sodium and potassium.

The invention also relates to a catalyst composite for the production of alkyl esters from a feedstock including one or more fatty acid glycerol esters or one or more fatty acids or mixture thereof, the catalyst composite comprises a catalyst composition comprising of oxides, mixed oxides, silicates or sulphates of two or more of silica, aluminium, iron, calcium, magnesium, sodium and potassium; and a nano composite catalyst having a particle size in the range of 5 nm to 1000 nm and comprising of oxides or mixed oxides of one or more of silica, alumina, calcium and iron.

### BRIEF DESCRIPTION OF ACCOMPANYING FIGURES

The accompanying drawing illustrates the preferred embodiments of the invention and together with the following detailed description serves to explain the principles of the invention.

FIG. 1 illustrates X-Ray Diffraction spectrum of a sample of fresh and re-used solid catalyst.

### DETAILED DESCRIPTION

To promote an understanding of the principles of the invention, reference will be made to the embodiment and specific language will be used to describe the same. It will nevertheless be understood that no limitation of scope of the invention is thereby intended, such alterations and further modifications in the illustrated process and such further applications of the principles of the inventions as illustrated therein being contemplated as would normally occur to one skilled in art to which the invention relates.

A catalyst for the production of alkyl esters from a feedstock including one or more fatty acid glycerol esters or one or more fatty acids or mixture thereof is described. The catalyst comprises of a nano composite catalyst having a particle size in the range of 5 nm to 1000 nm and comprising oxides or mixed oxides of one or more of silica, alumina, calcium and iron. The nano composite catalyst may comprise of 25 to 75 weight percent tricalcium silicate (Ca₃SiO₅), 10 to 40 weight percent dicalcium silicate (Ca₂SiO₄), 1 to 20 weight percent tricalcium aluminate (Ca₃Al₂O₆) and 1 to 20 weight percent tetracalcium aluminoferrite (Ca₄Al₂Fe₂O₁₀).

In accordance with an aspect the catalyst is a catalyst composite including the nano composite catalyst. The catalyst composite may also contain any inert or active component. The amount of nano composite in the catalyst composite is at least 5 weight percent.

In accordance with an alternate embodiment, the catalyst is a catalyst composite, the catalyst composite comprising of a catalyst composition comprising of two or more of oxides, mixed oxides, silicates or sulphates of two or more of silica, aluminium, iron, calcium, magnesium, sodium and potassium.

The catalyst composition may comprise of 25 to 75 weight percent tricalcium silicate (Ca₃SiO₅), 10 to 40 weight percent dicalcium silicate (Ca₂SiO₄), 1 to 20 weight percent tricalcium aluminate (Ca₃Al₂O₆) and 1 to 20 weight percent tetracalcium aluminoferrite (Ca₄Al₂Fe₂O₁₀). In accordance with an aspect, the catalyst composition may further comprise of one or more of 0 to 1.5 weight percent of MgO, 0 to 1.5 weight percent of Na₂O, 0 to 5.4 weight percent of K₂O or 0 to 10 weight percent hydrated calcium sulphate.

Alternatively, the catalyst composition may comprise of 14 to 23 weight percent of SiO₂, 3 to 6 weight percent of Al₂O₃, 2.50 to 6 weight percent of Fe₂O₃, 43 to 67 weight percent of CaO, 1 to 1.5 weight percent of MgO, 0 to 1.5 weight percent of Na₂O and 0 to 5.4 weight percent of K₂O.

In accordance with an aspect, the catalyst composite may also contain any inert or active component. The amount of catalyst composition in the catalyst composite material is at least 5 weight percent.

In accordance with an aspect catalyst composition may include cement including but not limited to ordinary cement, Portland cement, white cement, masonary cement, hydraulic and non-hydraulic cements or any other type of cement or their mixture. The composition of cement typically varies within the following composition by 14 to 23 weight percent of SiO₂, 3 to 6 weight percent of Al₂O₃, 2.50 to 6 weight percent of Fe₂O₃, 43 to 67 weight percent of CaO, 1 to 1.5 weight percent of MgO, 0 to 1.4 weight percent of SO₃, 0 to 1.5 weight percent of Na₂O and 0 to 5.4 weight percent of K₂O.

Cement is a dry powder commonly used as a “binder” material. Binder is a substance that sets or hardens independently and helps bind other materials together. The Portland cement, most commonly used type of cement, is made by heating limestone with small quantities of clay type materials to over 1200° C. in a kiln. The resulting hard substance, called ‘clinker’, is then ground with a small amount of gypsum into a powder to make Ordinary Portland Cement (often referred to as OPC).

In accordance with an alternate embodiment, the catalyst is a catalyst composite comprising of the catalyst composition and the nano composite catalyst, the catalyst composition comprising of oxides, mixed oxides, silicates or sulphates of two or more of silica, aluminium, iron, calcium, magnesium, sodium and potassium; and the nano composite catalyst having a particle size in the range of 5 nm to 1000 nm and comprising of oxides or mixed oxides of one or more of silica, alumina, calcium and iron.

The catalyst composition may comprise of 25 to 75 weight percent tricalcium silicate (Ca₃SiO₅), 10 to 40 weight percent dicalcium silicate (Ca₂SiO₄), 1 to 20 weight percent Tricalcium aluminate (Ca₃Al₂O₆) and 1 to 20 weight percent tetracalcium aluminoferrite (Ca₄Al₂Fe₂O₁₀). In accordance with an aspect, the catalyst composition may further comprise of one or more of 0 to 1.5 weight percent of MgO, 0 to 1.5 weight percent of Na₂O, 0 to 5.4 weight percent of K₂O or 0 to 10 weight percent hydrated calcium sulphate.

Alternatively, the catalyst composition may comprise of 14 to 23 weight percent of SiO₂, 3 to 6 weight percent of Al₂O₃, 2.50 to 6 weight percent of Fe₂O₃, 43 to 67 weight percent of CaO, 1 to 1.5 weight percent of MgO, 0 to 1.5 weight percent of Na₂O, and 0 to 5.4 weight percent K₂O.

The nano composite catalyst may comprise of 25 to 75 weight percent tricalcium silicate (Ca₃SiO₅), 10 to 40 weight percent dicalcium silicate (Ca₂SiO₄), 1 to 20 weight percent tricalcium aluminate (Ca₃Al₂O₆) and 1 to 20 weight percent tetracalcium aluminoferrite (Ca₄Al₂Fe₂O₁₀).

The catalyst catalyses the transesterification of the fatty acid glycerol esters present in the feedstock as illustrated in the exemplified reaction below:

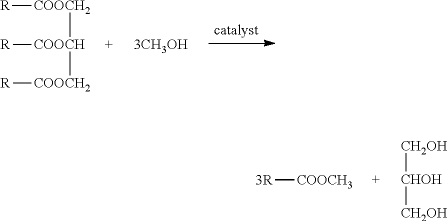

The catalyst also catalyzes the esterification of fatty acids present in the feedstock as illustrated in the exemplified reaction

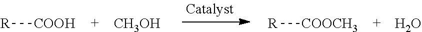

The process for production of alkyl esters comprises reacting a feedstock that includes one or more fatty acid glycerol esters or one or more fatty acids or mixture thereof with an alcohol at a temperature substantially 100° C. or more, in the presence of catalyst, to get a reaction mixture, the catalyst including a catalyst composition comprising of oxides, mixed oxides, silicates or sulphates of two or more of silica, aluminium, iron, calcium, magnesium, sodium and potassium. The reaction mixture contains a mixture of alkyl esters, alcohol and catalyst and alkyl esters are recovered from the reaction mixture.

In accordance with an aspect, the catalyst composition comprises of 25 to 75 weight percent tricalcium silicate (Ca₃SiO₅), 10 to 40 weight percent dicalcium silicate (Ca₂SiO₄), 1 to 20 weight percent tricalcium aluminate (Ca₃Al₂O₆) and 1 to 20 weight percent tetracalcium aluminoferrite (Ca₄Al₂Fe₂O₁₀). In accordance with an aspect, the catalyst composition may further comprise of one or more of 0 to 1.5 weight percent of MgO, 0 to 1.5 weight percent of Na₂O and 0 to 5.4 weight percent of K₂O or 0 to 10 weight percent hydrated calcium sulphate. Alternatively, the catalyst composition may comprise 14 to 23 weight percent of SiO₂, 3 to 6 weight percent of Al₂O₃, 2.50 to 6 weight percent of Fe₂O₃, 43 to 67 weight percent of CaO, 1 to 1.5 weight percent of MgO, 0 to 1.5 weight percent of Na₂O and 0 to 5.4 weight percent of K₂O.

The reaction may be carried out at a temperature in the range of 100° C. to 250° C. under autogenerated pressure. The reaction may also be carried out at high pressure of up to 50 bar.

Since alcohol and oils and fats have limited miscibility in each other, the reaction rates are naturally slow. However, elevated temperature and higher pressure, increases the solubility of the reaction mass (glycerol, monoglycerides, diaglycerides, fatty acid methyl esters) in alcohol. This increases the reaction rates and allows for an efficient reaction in terms of quantity of catalyst required and reaction time.

In accordance with an alternate embodiment, a process for production of alkyl esters using a nano composite catalyst is described. The process for production of alkyl esters comprises reacting a feedstock that includes one or more fatty acid glycerol esters or one or more fatty acids or mixture thereof with an alcohol in the presence of catalyst to get a reaction mixture, the catalyst including the nano composite catalyst having a particle size in the range of 5 nm to 1000 nm and comprising oxides of one or more of silica, alumina, calcium and iron. The reaction mixture contains a mixture of alkyl esters, alcohol and the catalyst including the nano composite catalyst and alkyl esters are recovered from the reaction mixture.

The nano composite catalyst comprises of one or more of Tricalcium silicate (Ca₃SiO₅), Calcium silicate (CaSiO₄), Tricalcium aluminate (Ca₃Al₂O₆) and Tetracalcium aluminoferrite (Ca₄Al₂Fe₂O₁₀). In accordance with an aspect, the composition of the nanocomposite varies within the range 25 to 75 weight percent tricalcium silicate (Ca₃SiO₅), 10 to 40 weight percent dicalcium silicate (Ca₂SiO₄), 1 to 20 weight percent tricalcium aluminate (Ca₃Al₂O₆) and 1 to 20 weight percent tetracalcium aluminoferrite (Ca₄Al₂Fe₂O₁₀).

In accordance with an alternate embodiment, the process for production of alkyl esters comprises reacting a feedstock that includes one or more fatty acid glycerol esters or one or more fatty acids or mixture thereof with an alcohol in the presence of catalyst, to get a reaction mixture, the catalyst being a catalyst composite, the catalyst composite comprising the catalyst composition and the nano composite catalyst, the catalyst composition comprising of oxides, mixed oxides, silicates or sulphates of two or more of silica, aluminium, iron, calcium, magnesium, sodium and potassium; and the nano composite catalyst having a particle size in the range of 5 nm to 1000 nm and comprising of oxides of one or more of silica, alumina, calcium and iron. The reaction mixture contains a mixture of alkyl esters, alcohol and the catalyst including the catalyst composite and alkyl esters are recovered from the reaction mixture.

The catalyst composition may comprise of 25 to 75 weight percent tricalcium silicate (Ca₃SiO₅), 10 to 40 weight percent dicalcium silicate (Ca₂SiO₄), 1 to 20 weight percent tricalcium aluminate (Ca₃Al₂O₆) and 1 to 20 weight percent tetracalcium aluminoferrite (Ca₄Al₂Fe₂O₁₀. In accordance with an aspect, the catalyst composition may further comprise of any one or more of 0 to 1.5 weight percent of MgO, 0 to 1.5 weight percent of Na₂O, 0 to 5.4 weight percent of K₂O or 0 to 10 weight percent hydrated calcium sulphate. Alternatively, the catalyst composition may comprise of 14 to 23 weight percent of SiO₂, 3 to 6 weight percent of Al₂O₃, 2.50 to 6 weight percent of Fe₂O₃, 43 to 67 weight percent of CaO, 1 to 1.5 weight percent of MgO, 0 to 1.5 weight percent of Na₂O and 0 to 5.4 weight percent of K₂O. The nano composite catalyst may comprise of one or more of Tricalcium silicate (Ca₃SiO₅), Calcium silicate (CaSiO₄), Tricalcium aluminate (Ca₃Al₂O₆) and Tetracalcium aluminoferrite (Ca₄Al₂Fe₂O₁₀). In accordance with an aspect, the composition of the nanocomposite varies within the range in 25 to 75 weight percent tricalcium silicate (Ca₃SiO₅), 10 to 40 weight percent dicalcium silicate (Ca₂SiO₄), 1 to 20 weight percent tricalcium aluminate (Ca₃Al₂O₆) and 1 to 20 weight percent tetracalcium aluminoferrite (Ca₄Al₂Fe₂O₁₀).

In accordance with an aspect the reaction is carried out in a temperature range of 60° C. to 200° C. under autogenerated pressure.

In accordance with an aspect, the production of alkyl esters comprises of reacting a feedstock that includes one or more fatty acid glycerol esters or one or more fatty acids or mixture thereof with an alcohol in the presence of catalyst, the catalyst including a catalyst composition comprising two or more of oxides, mixed oxides, silicates or sulphates of two or more of silica, aluminium, iron, calcium, magnesium, sodium and potassium at a temperature substantially 100° C. and autogenerated pressure for a predetermined period of time to get a reaction mixture, the reaction mixture contains a mixture of alkyl esters, glycerol, alcohol and catalyst; removing the catalyst from the said reaction mixture by filtration or any suitable conventional separation method to get a liquid with two phases, an alcohol containing alkyl esters rich layer and alcohol containing glycerol rich layer, separating the two phases and removing the alcohol from alkyl esters and glycerol rich liquids by conventional distillation to get alkyl esters and glycerol.

In accordance with an aspect, the production of alkyl esters comprises of reacting a feedstock that includes one or more fatty acid glycerol esters or one or more fatty acids or mixture thereof with an alcohol in the presence of catalyst, the catalyst including nano composite catalyst at an elevated temperature and autogenerated pressure for a predetermined period of time to get a reaction mixture, the reaction mixture contains a mixture of alkyl esters, glycerol, alcohol and catalyst including nano composite catalyst; removing the catalyst from the said reaction mixture by filtration or any suitable conventional separation method to get a liquid with two phases, an alcohol containing alkyl esters rich layer and alcohol containing glycerol rich layer, separating the two phases and removing the alcohol from alkyl esters and glycerol rich liquids by conventional distillation to get alkyl esters and glycerol.

In accordance with an aspect, the production of alkyl esters comprises of reacting a feedstock that includes one or more fatty acid glycerol esters or one or more fatty acids or mixture thereof with an alcohol in the presence of catalyst, the catalyst being a catalyst composite, the catalyst composite comprising a catalyst composition and a nano composite catalyst, the catalyst composition comprising oxides, mixed oxides, silicates or sulphates of two or more of silica, aluminium, iron, calcium, magnesium, sodium and potassium; and the nano composite catalyst having a particle size in the range of 5 nm to 1000 nm and comprising oxides of one or more of silica, alumina, calcium and iron at an elevated temperature and autogenerated pressure for a predetermined period of time to get a reaction mixture, the reaction mixture contains a mixture of alkyl esters, glycerol, alcohol and catalyst; removing the catalyst from the said reaction mixture by filtration or any suitable conventional separation method to get a liquid with two phases, an alcohol containing alkyl esters rich layer and alcohol containing glycerol rich layer, separating the two phases and removing the alcohol from alkyl esters and glycerol rich liquids by conventional distillation to get alkyl esters and glycerol.

In accordance with an aspect, a greater than 98% conversion is achieved by the process and a greater than 99% conversion is achieved using the catalyst under preferred reaction condition. As the catalyst does not dissolve in the reaction mixture, the quality of the biodiesel and glycerol obtained is purer than most conventional processes.

The catalyst is easily recovered from the reaction mixture by any method including gravitational settling, filtration, centrifugation or any combination thereof.

In accordance with an aspect, once separated, the catalyst may be re-used, if needed, as a catalyst for production of alkyl esters without any loss of catalytic activity. The recyclability has been tested for at least five cycles and the reaction proceeds with quantitative yield of the products.

In accordance with an aspect, the catalyst recovered from the reaction mixture may be washed and dried prior to reusing it as a catalyst for the production of alkyl esters. The catalyst recovered from the reaction mixture may be washed with any organic solvent in which the organics are soluble. In accordance with an aspect, hydroxylated solvents for example alcohols such as methanol and ethanol are used but less polar organic solvents like hydrocarbons (e.g., hexane) may also be used to selectively remove the ethyl esters. Glycerine left behind with the catalyst may be extracted with water or a hydroxylated solvent. Chlorinated solvents such as chloroform, dichloromethane may also be used.

The reaction mixture includes a layer containing fatty acid alkyl esters and alcohol and a layer containing glycerol and alcohol. Recovery of alkyl esters from the reaction mixture is carried out by separating the catalyst from the reaction mixture. The alkyl esters are recovered from the alkyl ester rich layer and separated from the glycerol rich lower layer and alcohol is removed from the two layers. Alternatively, alcohol can be distilled off by simply de-pressuring the reactor at the reaction temperature leaving behind two immiscible liquids in fatty acid alkyl ester and glycerol along with the catalyst.

In accordance with an aspect, the alcohol containing alkyl ester rich layer may be separated from the alcohol containing glycerol rich layer by any method including but not limited to gravitational settling, centrifugation, distillation, using separation funnel or a combination thereof. In accordance with an embodiment alcohol is removed from alkyl esters and glycerol by vacuum distillation.

The quantity of catalyst required is in the range of 1 to 30 weight percent with respect to the feedstock comprising fatty acid glycerol esters or free fatty acids or mixture thereof. Preferably the quantity of catalyst required is in the range of 2 and 25 weight percent with respect to the feed stock, and most preferably the quantity of catalyst required is in the range of 5 to 10 weight percent with respect to the feed stock.

In accordance with an aspect if less than 5 weight percent of catalyst is used the reaction is carried out at a temperature higher than 160° C. to achieve >99% conversion. It is also observed that the molar ratio of feedstock to alcohol is reduced. In accordance with an aspect, the molar ratio of the feedstock to alcohol may be in the range of 3 to 30, or preferably in the range of 7 to 15.

The feed stock used for this process may contain free fatty acids or fatty acid glycerol esters or mixture thereof. The fatty acid glycerol esters may be mono-, di- or tri-ester of glycerol with varying degree of unsaturation in the fatty acid chain. The feedstock used for the production of alkyl esters may be any fatty acid rich material including but not limited to vegetable oil, used vegetable oil, restaurant waste grease, acid oil or surplus liquid or solid fats such as vegetable shortening, surplus margarine or animal fats. Each of these may be used individually or as a mixture.

In accordance with an aspect, additional processing such as removal of excess water or filtering out of precipitate may be required before using animal fat or vegetable oil for this process.

The alcohol to be used for the reaction may be any C₁ to C₄ alcohol, including but not limited to methanol, ethanol, propa(en)nol and buta(en)nol. The alcohol used can be primary, secondary or tertiary in nature. Single alcohol or a mixture of two or more alcohols may also be used for the reaction.

Specific Embodiments are Described Below:

A process for producing alkyl esters, the process comprising reacting a feedstock that includes one or more fatty acid glycerol esters or one or more fatty acid or mixture thereof with a C₁ to C₄ alcohol in the presence of a catalyst at a temperature substantially 100° C. or more, the catalyst including a catalyst composition comprising oxides, mixed oxides, silicates or sulphates of two or more of silica, aluminium, iron, calcium, magnesium, sodium and potassium.

Such process(s) wherein the catalyst composition comprises of 25 to 75 weight percent tricalcium silicate (Ca₃SiO₅), 10 to 40 weight percent dicalcium silicate (Ca₂SiO₄), 1 to 20 weight percent tricalcium aluminate (Ca₃Al₂O₆) and 1 to 20 weight percent tetracalcium aluminoferrite (Ca₄Al₂Fe₂O₁₀).

Such process(s) wherein the catalyst composition further comprises of one or more of 0 to 1.5 weight percent of MgO, 0 to 1.5 weight percent of Na₂O, 0 to 5.4 weight percent of K₂O or 0 to 10 weight percent hydrated calcium sulphate.

Such process(s) wherein the catalyst composition comprises of 14 to 23 weight percent of SiO₂, 3 to 6 weight percent of Al₂O₃, 2.50 to 6 weight percent of Fe₂O₃, 43 to 67 weight percent of CaO, 1 to 1.5 weight percent of MgO, 0 to 1.5 weight percent of Na₂O and 0 to 5.4 weight percent of K₂O.

Further Specific Embodiments are Described Below:

A process for producing alkyl esters, the process comprising reacting a feedstock that includes one or more fatty acid glycerol ester or one or more fatty acid or mixture thereof with a C₁ to C₄ alcohol in the presence of a catalyst, the catalyst including a nano composite catalyst having a particle size in the range of 5 nm to 1000 nm and comprising of oxides or mixed oxides of one or more of silica, alumina, calcium and iron.

Such process(s) wherein the nano composite catalyst comprises of 25 to 75 weight percent tricalcium silicate (Ca₃SiO₅), 10 to 40 weight percent dicalcium silicate (Ca₂SiO₄), 1 to 20 weight percent tricalcium aluminate (Ca₃Al₂O₆) and 1 to 20 weight percent tetracalcium aluminoferrite (Ca₄Al₂Fe₂O₁₀).

Further Specific Embodiments are Described Below:

A process for producing alkyl esters, the process comprising reacting a feedstock that includes one or more fatty acid glycerol esters or one or more fatty acid or mixture thereof with a C₁ to C₄ alcohol in the presence of a catalyst, the catalyst being a catalyst composite comprising of a catalyst composition comprising of oxides, mixed oxides, silicates or sulphates of two or more of silica, aluminium, iron, calcium, magnesium, sodium and potassium; and a nano composite catalyst having a particle size in the range of 5 nm to 1000 nm and comprising of oxides or mixed oxides of one or more of silica, alumina, calcium and iron.

Such process(s) wherein the catalyst composition comprises of 25 to 75 weight percent tricalcium silicate (Ca₃SiO₅), 10 to 40 weight percent dicalcium silicate (Ca₂SiO₄), 1 to 20 weight percent Tricalcium aluminate (Ca₃Al₂O₆) and 1 to 20 weight percent tetracalcium aluminoferrite (Ca₄Al₂Fe₂O₁₀).

Such process(s) wherein the catalyst composition further comprises of one or more of 0 to 1.5 weight percent of MgO, 0 to 1.5 weight percent of Na₂O, 0 to 5.4 weight percent of K₂O or 0 to 10 weight percent hydrated calcium sulphate.

Such process(s) wherein the catalyst composition comprises of 14 to 23 weight percent of SiO₂, 3 to 6 weight percent of Al₂O₃, 2.50 to 6 weight percent of Fe₂O₃, 43 to 67 weight percent of CaO, 1 to 1.5 weight percent of MgO, 0 to 1.5 weight percent of Na₂O and 0 to 5.4 weight percent of K₂O.

Such process(s) wherein the nano composite catalyst comprises of 25 to 75 weight percent tricalcium silicate (Ca₃SiO₅), 10 to 40 weight percent dicalcium silicate (Ca₂SiO₄), 1 to 20 weight percent tricalcium aluminate (Ca₃Al₂O₆) and 1 to 20 weight percent tetracalcium aluminoferrite (Ca₄Al₂Fe₂O₁₀).

Such process(s) wherein the reaction is carried out at a temperature in the range of 60° C. to 200° C.

Such process(s) wherein the amount of catalyst used is the range of 1 to 30 weight percent with respect to the feedstock.

Such process(s) wherein the molar ratio of alcohol to feedstock is not more than 30.

Such process(s) wherein the process further comprises of recovering the catalyst from the reaction mixture.

Such process(s) wherein the process further comprises of separating the catalyst from the reaction mixture; washing and dying the catalyst; and reusing the catalyst for producing alkyl esters.

Such process(s) wherein the fatty acid ester is a mono-, di- or tri-ester of glycerol with varied degree of unsaturation in the fatty acid chain.

Such process(s) wherein the alcohol is any of methanol, ethanol, propen(en)ol or butan(en)ol or their mixtures.

Alkyl esters obtained by such process(s).

Further Specific Embodiments are Described Below:

A catalyst composite for the production of alkyl esters from a feedstock including one or more fatty acid glycerol esters or one or more fatty acids or mixture thereof, wherein the catalyst composite comprises of a nano composite catalyst having a particle size in the range of 5 nm to 1000 nm and comprising of oxides or mixed of one or more of silica, alumina, calcium and iron.

Such catalyst composite(s) wherein the nano composite catalyst comprises 25 to 75 weight percent tricalcium silicate (Ca₃SiO₅), 10 to 40 weight percent dicalcium silicate (Ca₂SiO₄), 1 to 20 weight percent tricalcium aluminate (Ca₃Al₂O₆) and 1 to 20 weight percent tetracalcium aluminoferrite (Ca₄Al₂Fe₂O₁₀).

Such catalyst composite(s) wherein the amount of nano composite catalyst in the catalyst composite is at least 5 weight percent and the remaining may be any inert or active component in the catalyst composite.

Further Specific Embodiments are Described Below:

A catalyst composite for the production of alkyl esters from a feedstock including one or more fatty acid glycerol esters or one or more fatty acids or mixture thereof, wherein the catalyst composite comprises a catalyst composition comprising of oxides, mixed oxides, silicates or sulphates of two or more of silica, aluminium, iron, calcium, magnesium, sodium and potassium.

Such catalyst(s) wherein the catalyst composition comprises of 25 to 75 weight percent tricalcium silicate (Ca₃SiO₅), 10 to 40 weight percent dicalcium silicate (Ca₂SiO₄), 1 to 20 weight percent Tricalcium aluminate (Ca₃Al₂O₆) and 1 to 20 weight percent tetracalcium aluminoferrite (Ca₄Al₂Fe₂O₁₀).

Such catalyst(s) wherein the catalyst composition further comprises of one or more of 0 to 1.5 weight percent of MgO, 0 to 1.5 weight percent of Na₂O, 0 to 5.4 weight percent of K₂O or 0 to 10 weight percent hydrated calcium sulphate.

Such catalyst composite(s) wherein the catalyst composition comprises of 14 to 23 weight percent of SiO₂, 3 to 6 weight percent of Al₂O₃, 2.50 to 6 weight percent of Fe₂O₃, 43 to 67 weight percent of CaO, 1 to 1.5 weight percent of MgO, 0 to 1.5 weight percent of Na₂O and 0 to 5.4 weight percent of K₂O.

Such catalyst composite(s) wherein the amount of catalyst composition in the catalyst composite is at least 5 weight percent and the remaining may be any inert or active component in the catalyst composite.

Such catalyst composite(s) wherein the catalyst composition comprises of cement.

Such catalyst composite(s) wherein the cement is any of Portland cement, white cement, masonary cement, hydraulic and non-hydraulic cements or mixture thereof.

Further Specific Embodiments are Described Below:

A catalyst composite for the production of alkyl esters from a feedstock including one or more fatty acid glycerol esters or one or more fatty acids or mixture thereof, the catalyst composite comprises a catalyst composition comprising of oxides, mixed oxides, silicates or sulphates of two or more of silica, aluminium, iron, calcium, magnesium, sodium and potassium; and a nano composite catalyst having a particle size in the range of 5 nm to 1000 nm and comprising of oxides or mixed oxides of one or more of silica, alumina, calcium and iron.

Such catalyst(s) wherein the catalyst composition comprises of 25 to 75 weight percent tricalcium silicate (Ca₃SiO₅), 10 to 40 weight percent dicalcium silicate (Ca₂SiO₄), 1 to 20 weight percent tricalcium aluminate (Ca₃Al₂O₆) and 1 to 20 weight percent tetracalcium aluminoferrite (Ca₄Al₂Fe₂O₁₀).

Such catalyst composite(s) wherein the catalyst composition further comprises of one or more of 0 to 1.5 weight percent of MgO, 0 to 1.5 weight percent of Na₂O, 0 to 5.4 weight percent of K₂O or 0 to 10 weight percent hydrated calcium sulphate.

Such catalyst composite(s) wherein the catalyst composition comprises of 14 to 23 weight percent of SiO₂, 3 to 6 weight percent of Al₂O₃, 2.50 to 6 weight percent of Fe₂O₃, 43 to 67 weight percent of CaO, 1 to 1.5 weight percent of MgO, 0 to 1.5 weight percent of Na₂O and 0 to 5.4 weight percent of K₂O.

Such catalyst composite(s) wherein the nano composite catalyst comprises of 25 to 75 weight percent tricalcium silicate (Ca₃SiO₅), 10 to 40 weight percent dicalcium silicate (Ca₂SiO₄), 1 to 20 weight percent tricalcium aluminate (Ca₃Al₂O₆) and 1 to 20 weight percent tetracalcium aluminoferrite (Ca₄Al₂Fe₂O₁₀).

The following examples are provided to explain and illustrate certain preferred embodiments of the process of the invention.

### Example 1

300 g of soyabean oil, 150 g of methanol and 30 g of catalyst, were put in batch reactor maintained at different reaction temperatures in the range of 120 to 230° C. under autogenerated pressure for 6 h after attaining the set temperature, the catalyst including a catalyst composition comprising 14 to 23 weight percent of SiO₂, 3 to 6 weight percent of Al₂O₃, 2.50 to 6 weight percent of Fe₂O₃, 43 to 67 weight percent of CaO, 1 to 1.5 weight percent of MgO, 0 to 1.5 weight percent of Na₂O and 0 to 5.4 weight percent of K₂O. At the end of this time, the reaction mass was allowed to reach room temperature, product mixture drained and catalyst filtered off. Two layers of liquid were present, upper one containing diesel in methanol and glycerol is present in lower layer along with methanol. Two layers of liquid were separated using separation funnel. Methanol was removed by distillation from both the layers separately. Similarly, methanol was distilled off by just de-pressurising the reactor and allowing the reminders of the reactor to settle down into two distinct layers and separating the solid catalyst by filtration. In either case conversions as high as >98% could be achieved using the catalyst. The products of the reactions were analyzed by the standard ASTM D 6584 method (Gas Chromatography method) protocols using silylating agent to derivatize the reaction components to be quantified. Reproducibility of results and comparison of the percent conversions were easily and routinely performed. The vegetable oil conversion in to ethyl esters (biodiesel) at 120° C. and 150° C. was 70% and 85% respectively. However at above 170° C., complete feed stock conversion (99%+) could be achieved. Similar experiments were performed to get feed stock conversion above 99% using a catalyst composite (10 weight percent commercial white cement and 2 weight percent nano composite).

### Example 2

300 g of pongamia oil, 150 g of methanol and 30 g of catalyst were put in batch reactor maintained at 210° C. under autogenerated pressure for 3 h, the catalyst including a catalyst composition comprising 14 to 23 weight percent of SiO₂, 3 to 6 weight percent of Al₂O₃, 2.50 to 6 weight percent of Fe₂O₃, 43 to 67 weight percent of CaO, 1 to 1.5 weight percent of MgO, 0 to 1.5 weight percent of Na₂O and 0 to 5.4 weight percent of K₂O. At the end of this time, the reaction mass was allowed to reach room temperature, product mixture drained and catalyst filtered off. Two layers of liquid were present, upper one containing ethyl esters in methanol and glycerol is present in lower layer along with methanol. Two layers of liquid were separated using a separation funnel. Methanol was removed by vacuum distillation from both the layers separately. More than 98% conversion into ethyl esters (biodiesel) was achieved with complete utilization of the triglycerides. However, the biodiesel obtained by this method had coloration since the starting material was also highly coloured. Similar experiments were performed to get feed stock conversion above 99% using a catalyst composite (10 weight percent commercial white cement and 2 weight % nano composite).

### Example 3

300 g of pongamia oil, 150 g of methanol and 30 g of catalyst were put in batch reactor maintained at 210° C. under autogenerated pressure for 3 h, the catalyst including a catalyst composition comprising 14 to 23 weight percent of SiO₂, 3 to 6 weight percent of Al₂O₃, 2.50 to 6 weight percent of Fe₂O₃, 43 to 67 weight percent of CaO, 1 to 1.5 weight percent of MgO, 0 to 1.5 weight percent of Na₂O and 0 to 5.4 weight percent of K₂O. At the end of this time, the reaction mass was allowed to reach room temperature, product mixture drained and catalyst filtered off. The separation was performed as described in example 1. Complete conversion of the triglycerides were seen with more than >98% yield of the ethyl esters (biodiesel).

### Example 4

300 g of feed stock (containing 50 weight % of free fatty acid and 50 weight % soybean oil), 150 g of methanol and 30 g of catalyst were put in a batch reactor maintained at 180° C. under autogenerated pressure for 3 h, the catalyst including a catalyst composition comprising 14 to 23 weight percent of SiO₂, 3 to 6 weight percent of Al₂O₃, 2.50 to 6 weight percent of Fe₂O₃, 43 to 67 weight percent of CaO, 1 to 1.5 weight percent of MgO, 0 to 1.5 weight percent of Na₂O and 0 to 5.4 weight percent of K₂O. At the end of this time, the excess methanol was distilled off by simply depressurising, as explained in example 1 and the two layers of liquid present in the reactor were separated. More than 95% yields of fatty acid methyl ester (FAME) and quantitative yields of glycerol were obtained.

### Example 5

300 g of pongamia oil, 150 g of methanol and 30 g of catalyst were simultaneously put in batch reactor maintained at 180° C. under autogenerated pressure, the catalyst including a catalyst composition comprising 14 to 23 weight percent of SiO₂, 3 to 6 weight percent of Al₂O₃, 2.50 to 6 weight percent of Fe2O3, 43 to 67 weight percent of CaO, 1 to 1.5 weight percent of MgO, 0 to 1.5 weight percent of Na₂O and 0 to 5.4 weight percent of K₂O. After every 30 minutes from the start of the reaction that is after attaining the set temperature, a small portion of the reaction mass was withdrawn and analyzed by Gas Chormatography method using standard ASTM protocols as described before. At the end of two hours, the reaction mass was allowed to reach room temperature, product mixture drained and catalyst filtered off. Two layers of liquid were present, upper one containing ethyl esters (bio-diesel) in methanol and glycerol is present in lower layer long with methanol. Two layers of liquid were separated using separation funnel. Table 1 depicts the rate of completion of the reaction using 10 weight % of the catalyst.

TABLE 1
Percent conversion of the triglyceride with time using 10 weight
% of catalyst.

| S. No. | Time in Minutes | percent conversion of triglyceride |
|---|---|---|
| 1 | 30 | 80 |
| 2 | 60 | 90 |
| 3 | 90 | 93 |
| 4 | 120 | >98 |
| 5 | 150 | >99 |
| 6 | 180 | >99 |

### Example 6

300 g of pongamia oil, 150 g of methanol and various amounts of catalysts (1-15 weight percent) were put in batch reactor maintained at 180° C. under autogenerated pressure for different time periods, the catalyst including a catalyst composition comprising 14 to 23 weight percent of SiO₂, 3 to 6 weight percent of Al₂O₃, 2.50 to 6 weight percent of Fe₂O₃, 43 to 67 weight percent of CaO, 1 to 1.5 weight percent of MgO, 0 to 1.5 weight percent of Na₂O and 0 to 5.4 weight percent of K₂O. At the end of this time, the reaction mass separated into individual fractions and percent conversions are recorded as shown in Table 2. Although, still higher amount of catalyst (say 20% and 30%) can easily be used but this not preferred as it may pose problems in down steam processing.

TABLE 2
Transesterification of pongamia oil using different amount of catalyst.

| S. No. | Catalyst | percent conversion |
|---|---|---|
| 1 | 1 | 85 |
| 2 | 5 | 98 |
| 3 | 7.5 | 99 |
| 4 | 10 | >99 |
| 5 | 15 | >99 |

### Example 7

This example illustrates the effect of methanol to soyabean oil molar ratio, which was varied between 4.5 to 45. While at a methanol to vegetable oil molar ratio of 7.5 and above, almost complete conversion of feed stock was obtained in about 2 to 4 hours at 180° C. At a lower methanol content (methanol to feedstock molar ratio equal or less that 4.5), about 90% feedstock conversion to ethyl esters (biodiesel) was observed.

Alcohols including ethanol, propanol and butanol were all tried and they resulted in the formation of fatty acid methyl ester (FAME) with the corresponding alcohol components in them in quantitative yields when the alcohol to oil molar ratio was 15:1.

### Example 8

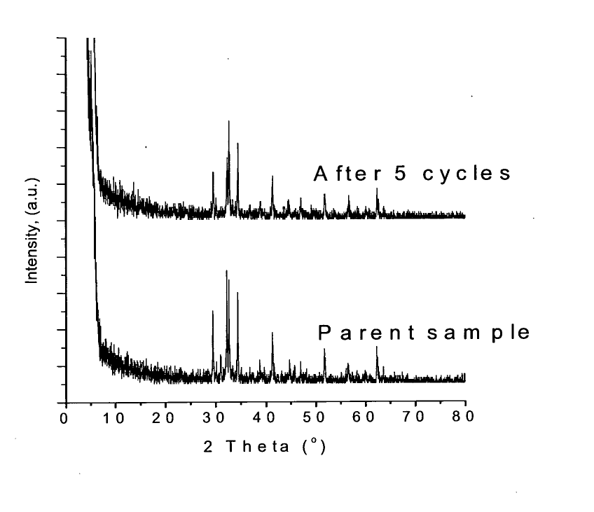

Experiment as depicted in example 1 was repeated with the re-cycled catalyst. The catalytic reactions were repeated for Soybean oil for more than 5 times and no reduction in the catalytic activity was seen indicating no or very small leaching of the metal into the reaction mass. This was also evident from the X-Ray Diffraction spectrum of a sample of the fresh and re-used Portland catalyst as illustrated in FIG. 1.

### Example 9

150 g of methanol and 30 g of the catalyst was treated at 180° C. for 3 hours under autogeneous pressure in a closed reactor. After this time the reaction mass was allowed to cool down and the catalyst was separated from the liquid. This liquid thus obtained was reacted with 300 g of feed stock at 180° C. for 4 hours under autogenous pressure in a closed reactor. Similarly, as a controlled experiment, 150 g of pure methanol was reacted with 300 g of feed stock under same conditions. It was observed that that in both cases around 30% conversion of feed stock was obtained. Further, the methanol treated solid material, as obtained from the above reaction with methanol was used as catalyst, where 150 g fresh methanol and 300 g feed stock were reacted at 180° C. for 4 hours, where more than 99% conversion of feed stock was observed. This demonstrates that the reaction is substantially carried out by solid catalyst under heterogeneous conditions.

### INDUSTRIAL APPLICABILITY

The process as described produces biodiesel in an economically efficient and an environmental friendly manner. As the catalyst is a solid catalyst, it can be easily separated from the reaction mixture and re-used thereby eliminating the need of neutralization step and aqueous washes that are associated with use of conventional catalysts. Moreover, as the catalyst catalyses both the esterification reaction of the free fatty acids and the transesterification of triglycerides that are present in the fatty acid starting material (free fatty acids and, oils and fats) the process has several advantages. Firstly, the efficiency of the process increases since no acid pre-treatment process and subsequent neutralization steps are needed. Also, alkyl esters (biodiesel) along with glycerine are generated as the only reaction product without any contaminations. This enables easy separation of the two immiscible layers from the catalyst, yielding biodiesel in quantitative yield that needs no further purification. The contaminations can only come from such sources where the free acid contents are higher than 20 weight percent in the oil, as seen in the case of acid oil. The catalyst separated from the reaction mixture does not lose its catalytic activity and may be reused as a catalyst, thereby reducing the cost of biodiesel production.

The embodiments of the invention, described above, are intended to be exemplary, and not limiting. Many variations are possible, within the scope of the invention. These and other modifications are to be deemed within the spirit and scope of the following claims.

1. A process for producing alkyl esters, the process comprising reacting a feedstock that includes one or more fatty acid glycerol esters or one or more fatty acid or mixture thereof with a C₁ to C₄ alcohol in the presence of a heterogeneous catalyst at a temperature substantially 100° C. or more, the catalyst including a catalyst composition comprising oxides or mixed oxides of silica, aluminium and calcium wherein the amount of catalyst used is substantially in the range of 1 to 30 weight percent with respect to the feedstock.

2. A process for producing alkyl esters, the process comprising reacting a feedstock that includes one or more fatty acid glycerol ester or one or more fatty acid or mixture thereof with a C₁ to C₄ alcohol in the presence of a catalyst, the catalyst including a nano composite catalyst having a particle size in the range of 5 nm to 1000 nm and comprising of oxides or mixed oxides of silica, alumina and calcium.

3. A process for producing alkyl esters, the process comprising reacting a feedstock that includes one or more fatty acid glycerol esters or one or more fatty acid or mixture thereof with a C₁ to C₄ alcohol in the presence of a catalyst, the catalyst being a catalyst composite comprising of:
a catalyst composition comprising of oxides or mixed oxides of silica, aluminium, and calcium; and
a nano composite catalyst having a particle size in the range of 5 nm to 1000 nm and comprising of oxides or mixed oxides of silica, alumina and calcium.

4. A process as claimed in claim 1, wherein the catalyst composition comprises of 25 to 75 weight percent tricalcium silicate (Ca₃SiO₅), 10 to 40 weight percent dicalcium silicate (Ca₂SiO₄), 1 to 20 weight percent tricalcium aluminate (Ca₃Al₂O₆) and 1 to 20 weight percent tetracalcium aluminoferrite (Ca₄Al₂Fe₂O₁₀).

5. A process as claimed in claim 4, wherein the catalyst composition further comprises of one or more of 0 to 1.5 weight percent of MgO, 0 to 1.5 weight percent of Na₂O, 0 to 5.4 weight percent of K₂O or 0 to 10 weight percent hydrated calcium sulphate.

6. A process as claimed in claim 1, wherein the catalyst composition comprises of 14 to 23 weight percent of SiO₂, 3 to 6 weight percent of Al₂O₃, 2.50 to 6 weight percent of Fe₂O₃, 43 to 67 weight percent of CaO, 1 to 1.5 weight percent of MgO, 0 to 1.5 weight percent of Na₂O and 0 to 5.4 weight percent of K₂O.

7. A process as claimed in claim 2, wherein the nano composite catalyst comprises of 25 to 75 weight percent tricalcium silicate (Ca₃SiO₅), 10 to 40 weight percent dicalcium silicate (Ca₂SiO₄), 1 to 20 weight percent tricalcium aluminate (Ca₃Al₂O₆) and 1 to 20 weight percent tetracalcium aluminoferrite (Ca₄Al₂Fe₂O₁₀.

8. A process as claimed in claim 2, wherein the reaction is carried out at a temperature in the range of 60° C. to 200° C.

9. (canceled)

10. A process as claimed in claim 1, wherein the molar ratio of alcohol to feedstock is not more than 30.

11. A process as claimed in claim 1, wherein the process further comprises of recovering the catalyst from the reaction mixture; washing and drying the catalyst; and reusing the catalyst for producing alkyl esters.

12.-14. (canceled)

15. Alkyl esters obtained by a process as claimed in claim 1.

16. A catalyst composite for the production of alkyl esters from a feedstock including one or more fatty acid glycerol esters or one or more fatty acids or mixture thereof, wherein the catalyst composite comprises of a nano composite catalyst having a particle size in the range of 5 nm to 1000 nm and comprising of oxides or mixed oxides of silica, alumina and calcium.

17.-21. (canceled)

22. A catalyst composite for the production of alkyl esters from a feedstock including one or more fatty acid glycerol esters or one or more fatty acids or mixture thereof, the catalyst composite comprises:
a catalyst composition comprising of oxides or mixed oxides of silica, aluminium and calcium; and
a nano composite catalyst having a particle size in the range of 5 nm to 1000 nm and comprising of oxides or mixed oxides of silica, alumina and calcium.

23. A catalyst composite as claimed in claim 16, wherein the nano composite catalyst comprises of 25 to 75 weight percent tricalcium silicate (Ca₃SiO₅), 10 to 40 weight percent dicalcium silicate (Ca₂SiO₄), 1 to 20 weight percent tricalcium aluminate (Ca₃Al₂O₆) and 1 to 20 weight percent tetracalcium aluminoferrite (Ca₄Al₂Fe₂O₁₀).

24. A catalyst composite as claimed in claim 22, wherein the catalyst composition comprises of 25 to 75 weight percent tricalcium silicate (Ca₃SiO₅), 10 to 40 weight percent dicalcium silicate (Ca₂SiO₄), 1 to 20 weight percent tricalcium aluminate (Ca₃Al₂O₆) and 1 to 20 weight percent tetracalcium aluminoferrite (Ca₄Al₂Fe₂O₁₀).

25. A catalyst composite as claimed in claim 24, wherein the catalyst composition further comprises of one or more of 0 to 1.5 weight percent of MgO, 0 to 1.5 weight percent of Na₂O, 0 to 5.4 weight percent of K₂O or 0 to 10 weight percent hydrated calcium sulphate.

26. A catalyst composite as claimed in claim 22, wherein the catalyst composition comprises of 14 to 23 weight percent of SiO₂, 3 to 6 weight percent of Al₂O₃, 2.50 to 6 weight percent of Fe₂O₃, 43 to 67 weight percent of CaO, 1 to 1.5 weight percent of MgO, 0 to 1.5 weight percent of Na₂O and 0 to 5.4 weight percent of K₂O.

27.-28. (canceled)

29. A process as claimed in claim 3, wherein the catalyst composition comprises of 25 to 75 weight percent tricalcium silicate (Ca₃SiO₅), 10 to 40 weight percent dicalcium silicate (Ca₂SiO₄), 1 to 20 weight percent tricalcium aluminate (Ca₃Al₂O₆) and 1 to 20 weight percent tetracalcium aluminoferrite (Ca₄Al₂Fe₂O₁₀).

30. A process as claimed in claim 29, wherein the catalyst composition further comprises of one or more of 0 to 1.5 weight percent of MgO, 0 to 1.5 weight percent of Na₂O, 0 to 5.4 weight percent of K₂O or 0 to 10 weight percent hydrated calcium sulphate.

31. A process as claimed in claim 3, wherein the catalyst composition comprises of 14 to 23 weight percent of SiO₂, 3 to 6 weight percent of Al₂O₃, 2.50 to 6 weight percent of Fe₂O₃, 43 to 67 weight percent of CaO, 1 to 1.5 weight percent of MgO, 0 to 1.5 weight percent of Na₂O and 0 to 5.4 weight percent of K₂O.

32. A process as claimed in claim 3, wherein the nano composite catalyst comprises of 25 to 75 weight percent tricalcium silicate (Ca₃SiO₅), 10 to 40 weight percent dicalcium silicate (Ca₂SiO₄), 1 to 20 weight percent tricalcium aluminate (Ca₃Al₂O₆) and 1 to 20 weight percent tetracalcium aluminoferrite (Ca₄Al₂Fe₂O₁₀).

33. A process as claimed in claim 3, wherein the reaction is carried out at a temperature in the range of 60° C. to 200° C.

34. A process as claimed in claim 2, wherein the molar ratio of alcohol to feedstock is not more than 30.

35. A process as claimed in claim 3, wherein the molar ratio of alcohol to feedstock is not more than 30.

36. A process as claimed in claim 2, wherein the process further comprises of recovering the catalyst from the reaction mixture; washing and drying the catalyst; and reusing the catalyst for producing alkyl esters.

37. A process as claimed in claim 3, wherein the process further comprises of recovering the catalyst from the reaction mixture; washing and drying the catalyst; and reusing the catalyst for producing alkyl esters.

38. Alkyl esters obtained by a process as claimed in claim 2.

39. Alkyl esters obtained by a process as claimed in claim 3.

40. A catalyst composite as claimed in claim 22, wherein the nano composite catalyst comprises of 25 to 75 weight percent tricalcium silicate (Ca₃SiO₅), 10 to 40 weight percent dicalcium silicate (Ca₂SiO₄), 1 to 20 weight percent tricalcium aluminate (Ca₃Al₂O₆) and 1 to 20 weight percent tetracalcium aluminoferrite (Ca₄Al₂Fe₂O₁₀).
